(19) United States
(12) Patent Application Publication
(10) Pub. No.: US 2013/0053422 A1
(43) Pub. Date: Feb. 28, 2013

(54) Antimicrobial Compositions

(71) Applicants: Sarah L. Edmonds, Canal Fulton, OH (US); James Bingham, Akron, OH (US); Mitchell Cohen, Salisbury, NC (US); Helen Burk, Cincinnati, OH (US)

(72) Inventors: Sarah L. Edmonds, Canal Fulton, OH (US); James Bingham, Akron, OH (US); Mitchell Cohen, Salisbury, NC (US); Helen Burk, Cincinnati, OH (US)

(21) Appl. No.: 13/636,357

(22) PCT Filed: Mar. 22, 2011
(86) PCT No.: PCT/US11/29293

§ 371 Date: Nov. 5, 2012

Related U.S. Application Data
(60) Provisional application No. 61/316,575, filed on Mar. 23, 2010.

(51) Int. Cl. A01N 37/46; A61K 31/425; A01P 3/00; A61P 31/00; A01P 1/00; A61P 17/00; A61K 31/223; A01N 43/80

(52) U.S. Cl. 514/372; 514/551

(57) ABSTRACT

Environmentally beneficial antimicrobial compositions are described that include a cationic surfactant and certain antimicrobial agents or preservatives. Useful cationic surfactants include lauric arginate (LAE). Advantageously, the pH of the composition may be adjusted to reduce irritancy.

### RELATED APPLICATIONS

This application claims the benefit of U.S. Provisional Application No. 61/316,575, filed Mar. 23, 2010, which is incorporated herein by reference.

### TECHNICAL FIELD

One or more embodiments of the present invention provide antimicrobial compositions as well as a method for surface disinfection, where the method includes contacting the surface with a composition that includes lauric arginate (LAE) and one or more of select antimicrobial agents or preservatives.

### BACKGROUND OF THE INVENTION

There is a need to provide environmentally beneficial products for hand and skin sanitizers and surface disinfection. Rapid, broad spectrum efficacy is also needed.

Many products contain ingredients that can be harsh on skin, particularly with repeated use. Therefore, a need remains for highly efficacious products that are milder to the skin. Other improvements that are needed include products with reduced environmental impact.

Environmentally preferable products have less detrimental impact on the environment in terms of their raw materials, manufacturing, use, and disposal. Desirable improvements include products with reduced concentrations of synthetic ingredients, less flammability, and reduced volatile organic compounds.

Lauric arginate (LAE) is a food-grade cationic surfactant. It has been described as an effective preservative, and has been combined with various components for enhanced preservative effect. LAE is sometimes referred to as ethyl lauroyl arginate, lauric arginate ethyl ester, and lauramide arginine ethyl ester.

U.S. Pat. No. 7,074,447 describes a combination of LAE with potassium sorbate, calcium sorbate or sorbic acid, and a method of preserving food products.

U.S. Pat. No. 7,196,117 describes the use of LAE with an antimicrobial agent such as triclosan, phenoxyethanol or chlorhexidine gluconate (CHG) in deodorants and oral care.

U.S. Pat. No. 7,758,851 describes the use of LAE in preservative systems suitable for cosmetics.

U.S. Pat. App. Pub. No. 2009/0326031 describes the use of LAE for the treatment of virus infections, where nearly complete reduction of Herpes virus type 1 Vaccinia virus and bovine parainfluenzae 3 were observed after 5 to 60 minutes.

However, rapid, broad spectrum efficacy within an exposure time of 1 minute or less is needed for purposes of disinfecting hands, skin, and other surfaces.

### SUMMARY OF THE INVENTION

In one or more embodiments, the present invention provides a composition that includes a cationic surfactant and certain antimicrobial agents or preservatives.

In one or more embodiments, the present invention provides a composition that includes lauric arginate (LAE) and an antimicrobial agent or preservative selected from C₁₋₆ alcohols, C₆₋₁₀ 1,2-alkanediols, and mixtures thereof. Advantageously, the pH of the composition is adjusted to from about 3.5 to about 9.5.

In one or more embodiments, the present invention provides a method for surface disinfection, wherein the method includes contacting the surface with a composition that includes LAE and an antimicrobial agent or preservative selected from C₁₋₆ alcohols, C₆₋₁₀ 1,2-alkanediols, and mixtures thereof, the pH of the composition having been adjusted to from about 3.5 to about 9.5.

In one or more embodiments, the present invention provides a method for preparing an antimicrobial composition, wherein the method includes combining LAE and an antimicrobial agent or preservative selected from C₁₋₆ alcohols, and C₆₋₁₀ 1,2-alkanediols, and adjusting the pH of the composition to from about 3.5 to about 9.5.

### DETAILED DESCRIPTION OF ILLUSTRATIVE EMBODIMENTS

In one or more embodiments, the present method provides an environmentally preferable antimicrobial composition with broad spectrum efficacy and reduced skin irritancy. The physical form of the antimicrobial composition is not particularly limited, and in one or more embodiments, the composition may be presented as a liquid that is poured, pumped, sprayed, or otherwise dispensed, a gel, an aerosol, or a foam, including both aerosol and non-aerosol foams. In one or more embodiments, the antimicrobial composition may be presented as a wipe, i.e. a tissue or cloth that is wiped over a surface. In addition to being effective as a hand sanitizer, the antimicrobial composition of the present invention may be employed on a wide variety of surfaces or substrates, including skin, porous, and non-porous surfaces. The antimicrobial composition may be a leave-on or rinse-off product.

In one or more embodiments, the composition includes a cationic surfactant and an antimicrobial agent or preservative. Advantageously, it has been found that antimicrobial compositions comprising a cationic surfactant such as those derived from the condensation of fatty acids and esterified dibasic amino acids have increased efficacy against a broad spectrum of gram positive and gram negative bacteria, fungi, parasites, and viruses, particularly when an enhancer such as certain antimicrobial agents or preservatives is also present.

As described hereinbelow, it has been surprisingly found that efficacy is enhanced when the pH of the composition is increased to about 3.5 to about 9.

In one or more embodiments, the cationic surfactant is derived from the condensation of a fatty acid and an esterified dibasic amino acid. Advantageously, in certain embodiments the cationic surfactant may be prepared from naturally occurring substances.

In one or more embodiments, cationic surfactant may be represented by the formula

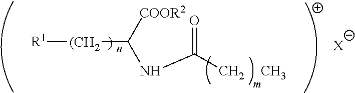

where R¹ is selected from

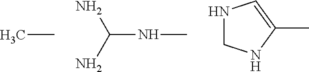

R² is an aromatic group or an alkyl group having from 1 to 18 carbon atoms, m is from about 8 to about 14, and n is from 0 to about 4.

In one or more embodiments, X is chloride, bromide, or a counter ion derived from an organic or inorganic acid or a phenolic compound. Examples of acids that may be the source of the counter ion X include acetic acid, citric acid, lactic acid, fumaric acid, maleic acid, gluconic acid, propionic acide, sorbic acid, benzoic acid, carbonic acid, glutamic acid, lauric acid, oleic acid, linoleic acid, phosphoric acid, nitric acid, sulfuric acid, and thiocyanic acid.

Examples of phenolic compounds that may be the source of the counter ion X include butylated hydroxyanisole (BHA), butylated hydroxytoluene, tertiary butylhydroquinone, methylparaben, ethylparaben, propylparaben, and butylparaben.

In one or more embodiments, the cationic surfactant is lauric arginate (LAE), which may be prepared from the naturally occurring substances L-arginine and lauric acid. LAE is commercially available, for example from Vedeqsa Inc. under the tradename Aminat.

The preparation of LAE has been described in the literature, for example in Spanish patent application ES-A-512643. The synthesis of cationic surfactants such as LAE has been further described in U.S. Pat. Nos. 5,780,658, 7,087,769, and 7,399,616, all of which are incorporated by reference herein.

In one embodiment, amount of cationic surfactant is at least about 0.02 wt. %, based upon the total weight of the antimicrobial composition, in another embodiment at least about 0.05, and in yet another embodiment at least about 0.1 wt. %, based upon the total weight of the antimicrobial composition. Generally, useful amounts of cationic surfactant are from about 0.02 to about 30 wt. %, based upon the total weight of the antimicrobial composition. In one embodiment, LAE is present in an amount of from about 0.02 to about 30 weight percent, in another embodiment, LAE is present in an amount of from about 0.05 to about 10 wt. %, in another embodiment, LAE is present in an amount of from about 0.1 to about 5 wt. %, in yet another embodiment, from about 0.15 to about 1 wt. %, and in still yet another embodiment, from about 0.2 to about 0.75 wt. %, based upon the total weight of the antimicrobial composition. It will be understood that greater amounts of cationic surfactant can be employed, if desired, and are expected to perform at least equally as well.

In certain embodiments, the cationic surfactant is added to the antimicrobial composition as a solution or emulsion. In other words, the cationic surfactant may be premixed with a carrier to form a solution or emulsion, with the proviso that the carrier does not deleteriously affect the sanitizing properties of the composition. Examples of carriers include water, alcohol, glycerol, glycols such as propylene or ethylene glycol, ketones, linear and/or cyclic hydrocarbons, triglycerides, carbonates, silicones, alkenes, esters such as acetates, benzoates, fatty esters, glyceryl esters, ethers, amides, polyethylene glycols and PEG/PPG copolymers, inorganic salt solutions such as saline, and mixtures thereof. Advantageously, the carrier may be selected from naturally derived compounds. It will be understood that, when the cationic surfactant is premixed to form a solution or emulsion, the amount of solution or emulsion that is added to the antimicrobial composition is selected so that the amount of cationic surfactant falls within the ranges set forth herein. In one embodiment, LAE is added to the antimicrobial composition together with glycerin as a carrier.

Certain antimicrobial agents and preservatives enhance the antimicrobial efficacy of LAE. Examples of enhancers include compounds selected from C₁₋₆ alcohols, C₆₋₁₂ 1,2-alkanediols, quaternary ammonium compounds, phenolic compounds, and 2-methyl-1,2-thiazol-3-one.

Therefore, in one or more embodiments, the antimicrobial composition comprises LAE and a C₁₋₆ alcohol. C₁₋₆ alcohols, i.e. alcohols containing 1 to 6 carbon atoms, are sometimes referred to as lower alkanols, and examples include methanol, ethanol, propanol, butanol, pentanol, hexanol, and isomers and mixtures thereof. In one embodiment, the C₁₋₆ alcohol comprises ethanol, propanol, or butanol, or isomers or mixtures thereof. In another embodiment, the C₁₋₆ alcohol comprises ethanol.

Generally, the antimicrobial composition comprises at least about 1 percent by weight (wt. %) C₁₋₆ alcohol, based upon the total weight of the antimicrobial composition. In one embodiment, the antimicrobial composition comprises at least about 2 weight percent C₁₋₆ alcohol, in another embodiment, the antimicrobial composition comprises at least about 10 weight percent C₁₋₆ alcohol, in another embodiment, the antimicrobial composition comprises at least about 20 weight percent C₁₋₆ alcohol, in another embodiment, the antimicrobial composition comprises at least about 40 weight percent C₁₋₆ alcohol, in another embodiment, the antimicrobial composition comprises at least about 50 weight percent C₁₋₆ alcohol, in another embodiment, the antimicrobial composition comprises at least about 60 weight percent C₁₋₆ alcohol, in another embodiment, the antimicrobial composition comprises at least about 65 weight percent C₁₋₆ alcohol, in yet another embodiment, the antimicrobial composition comprises at least about 70 weight percent C₁₋₆ alcohol, and in still yet another embodiment, the antimicrobial composition comprises at least about 78 weight percent C₁₋₆ alcohol, based upon the total weight of antimicrobial composition. More or less alcohol may be required in certain instances, depending particularly on other ingredients and/or the amounts thereof employed in the composition.

Advantageously, rapid and broad spectrum antimicrobial efficacy is observed at lower concentrations of alcohol when LAE is present compared to when LAE is not present. Thus, in certain embodiments the amount of alcohol may be significantly reduced, when compared with conventional antimicrobial compositions. In one or more embodiments, the antimicrobial composition comprises less about 90 weight percent alcohol, in other embodiments, the antimicrobial composition comprises less than about 60 weight percent alcohol, in other embodiments, the antimicrobial composition comprises less than about 50 weight percent alcohol, in yet other embodiments, the antimicrobial composition comprises less than about 40 weight percent alcohol, based upon the total weight of the antimicrobial composition. In one or more embodiments, the antimicrobial composition comprises LAE and from about 2 to about 20 wt. % ethanol.

In one or more embodiments, the antimicrobial composition comprises one or more C₆₋₁₀ alkane diols, i.e. diols having a carbon chain length of 6 to 10. In one or more embodiments, the diol includes 1,2-hexanediol, 1,2-octanediol, 1,9-nonanediol, 1,2-decanediol, 1,10-decanediol, or a mixture thereof. In one or more embodiments, the diol includes 1,2-hexanediol, 1,2-octanediol, or a mixture thereof 1,2-octanediol is sometimes referred to as caprylyl glycol. 1,2-decanediol is sometimes referred to as decylene glycol. In one or more embodiments, the antimicrobial composition comprises LAE and 1,2-octanediol. In one or more embodiments, the antimicrobial composition comprises LAE, from about 2 to about 20 wt. % ethanol, and 1,2-octanediol. Without wishing to be bound by theory, it is believed that the alkane diol enhances the rapid, broad spectrum efficacy of the cationic surfactant and/or the lower alkanol.

In one embodiment, an efficacy-enhancing amount of diol is at least about 0.02 wt. %, based upon the total weight of the antimicrobial composition, in another embodiment at least about 0.05, and in yet another embodiment at least about 0.1 wt. %, based upon the total weight of the antimicrobial composition.

Generally, an efficacy-enhancing amount of diol is from about 0.02 to about 10 wt. %, based upon the total weight of the antimicrobial composition. In one embodiment, the diol is present in an amount of from about 0.05 to about 5 weight percent, in another embodiment, the diol is present in an amount of from about 0.1 to about 1 wt. %, in yet another embodiment, from about 0.15 to about 0.8 wt. %, and in still yet another embodiment, from about 0.2 to about 0.75 wt. %, based upon the total weight of the antimicrobial composition. It will be understood that greater amounts of diol can be employed, if desired, and are expected to perform at least equally as well. In one embodiment, the antimicrobial composition comprises from about 0.02 to about 30 wt. % of C₆₋₁₀ alkane diol, based upon the total weight of the antimicrobial composition.

In certain embodiments, the diol is added to the antimicrobial composition as a solution or emulsion. In other words, the diol may be premixed with a carrier to form a diol solution or emulsion, with the proviso that the carrier does not deleteriously affect the sanitizing properties of the composition. Examples of carriers include water, alcohol, glycols such as propylene or ethylene glycol, ketones, linear and/or cyclic hydrocarbons, triglycerides, carbonates, silicones, alkenes, esters such as acetates, benzoates, fatty esters, glyceryl esters, ethers, amides, polyethylene glycols and PEG/PPG copolymers, inorganic salt solutions such as saline, and mixtures thereof. It will be understood that, when the diol is premixed to form a diol solution or emulsion, the amount of solution or emulsion that is added to the antimicrobial composition is selected so that the amount of diol falls within the ranges set forth hereinabove.

Advantageously, certain quaternary ammonium compounds enhance the efficacy of the antimicrobial compositions. Quaternary enhancers include quaternary ammonium compounds of the structure NR₄⁺ where R is an organic group. Exemplary quaternary ammonium antimicrobial agents that can be used according to the invention include quaternium-15, benzethonium chloride (BZT), benzalkonium chloride, methyl benzethonium chloride, and benzoxonium chloride.

In one or more embodiments, the quaternary ammonium compound comprises quaternium-15. Quaternium-15 is sometimes also referred to as 1-(3-Chloroallyl)-3,5,7-triaza-1-azoniaadamantane chloride, N-(3-chloroallyl) hexaminium chloride, hexamethylenetetramine chloroallyl chloride, 3,5,7-triaza-1-azoniaadamantane or 1-(3-chloroallyl)-chloride. It is commercially available, for example under the tradename Dowicil.

In one or more embodiments, the antimicrobial composition comprises LAE, at least one compound selected from the group consisting of C₁₋₆ alkanols, C₆₋₁₀ 1,2-alkanediols, and mixtures thereof, and further comprises quaternium-15.

In one or more embodiments, the antimicrobial composition comprises LAE and benzalkonium chloride, and optionally further comprises at least one of C₁₋₆ alkanols, C₆₋₁₀ 1,2-alkanediols, and mixtures thereof.

In one or more embodiments, an efficacy-enhancing amount of quaternary antimicrobial agent is from about 0.02 to about 0.2 wt. %, based upon the total weight of the antimicrobial composition.

Advantageously, certain phenolic antimicrobial agents and preservatives enhance the efficacy of the antimicrobial compositions. Phenolic antimicrobial agents include triclosan, chlorophenols (o-, m-, p-), 2,4-dichlorophenol, p-nitrophenol, picric acid, xylenol, p-chloro-m-xylenol, cresols (o-, m-, p-), p-chloro-m-cresol, pyrocatechol, resorcinol, 4-n-hexylresorcinol, pyrogallol, phloroglucin, carvacrol, thymol, p-chlorothymol, o-phenylphenol, o-benzylphenol, p-chloro-o-benzylphenol, phenol, 4-ethylphenol, 4-phenolsulfonic acid, hexachlorophene, tetrachlorophene, dichlorophene, 2,3-dihydroxy-5,5′-dichlorodiphenyl sulfide, 2,2′-dihydroxy-3,3′,5,5′-tetrachlorodiphenyl sulfide, 2,2′-dihydroxy-3,5′,5,5′,6,6′-hexachlorodiphenyl sulfide, and 3,3′-dibromo-5,5′-dichloro-2,2′-dihydroxydiphenylamine.

Phenolic preservatives include 2-phenoxyethanol, methylparaben, ethylparaben, propylparaben, butyl paraben, sodium methyl paraben, sodium propyl paraben, butylparaben and isobutylparaben. Combinations of phenolic preservatives are commercially available.

In one or more embodiments, the phenolic enhancer includes one or more of 2-phenoxyethanol, methylparaben, ethylparaben, and propylparaben. In one or more embodiments, an efficacy-enhancing amount of phenolic enhancer is from about 0.1 to about 0.3 wt. %, based upon the total weight of the antimicrobial composition.

In one or more embodiments, the enhancer comprises 2-methyl-1,2-thiazol-3-one. This antimicrobial agent is available commercially, for example from Rohm and Haas under the tradename Kathon.

In one or more embodiments, an efficacy-enhancing amount of 2-methyl-1,2-thiazol-3-one is from about 0.1 to about 0.3 wt. %, based upon the total weight of the antimicrobial composition.

LAE can be used in association with common antimicrobials, such as 2,4,4′-trichloro-T-hydroxy-diphenylether (triclosan), 3,4,4-trichlorocarbanilid (triclocarban), 2-phenoxyethanol, chlorhexidine salts (CHG), parachlormetaxylenol (PCMX), hexetidine and cetylpyridinium salts. On the other hand, antimicrobial agents such as triclosan, CHG, and PCMX can be irritants and are not generally regarded as natural ingredients. Advantageously these antimicrobial agents are not necessary according to the present invention. Thus, in one embodiment, the amount of each of 2,4,4′-trichloro-2′-hydroxy-diphenylether (triclosan), 3,4,4-trichlorocarbanilid (triclocarban), 2-phenoxyethanol, chlorhexidine salts (CHG), parachlormetaxylenol (PCMX), hexetidine and cetylpyridinium salts is less than about 0.1 wt. %, in another embodiment, less than about 0.05 wt. %, based upon the total weight of the antimicrobial composition. In another embodiment, the antimicrobial composition is devoid of 2,4,4′-trichloro-2′-hydroxy-diphenylether (triclosan), 3,4,4-trichlorocarbanilid (triclocarban), 2-phenoxyethanol, chlorhexidine salts (CHG), parachlormetaxylenol (PCMX), hexetidine and cetylpyridinium salts.

Surprisingly, it has been found that the antimicrobial activity of the combinations of LAE and certain antimicrobial agents and preservatives is higher than the activity displayed by the components when used alone at the same dosage. Therefore, amounts can be reduced from what is typically recommended based upon past studies. Because amounts of these antimicrobial agents can be reduced, the adverse toxic effects and/or irritation and/or allergy displayed by the antimicrobial systems may also be reduced. As explained hereinabove, products containing reduced amounts of synthetic materials are environmentally preferable, or “greener.”

The synergy seen with LAE was not predictable from the preservative effects that have been shown by LAE in the past. Other preservatives do not show this same synergy. For example, when LAE is combined with potassium sorbate or sodium benzoate, a decrease in antimicrobial efficacy is observed.

In one or more embodiments, the pH of the antimicrobial composition is from about 1.5 to about 10, in another embodiment from about 3.5 to about 9.5, in another embodiment from about 4.5 to about 9, in another embodiment from about 5 to about 8.5, in another embodiment from about 7 to about 8. Advantageously, and contrary to suggestions in the art for LAE-containing compositions, the pH of the antimicrobial composition can be adjusted from quite acidic to the more skin-friendly and neutral range of from about 5 to about 9, without loss of efficacy. In fact, in one or more embodiments, the efficacy improves when the pH of the antimicrobial composition is adjusted upward to from about 5 to about 9.

In one or more embodiments, the antimicrobial composition is formulated as a foamable composition. One or more foam agents may optionally be included in the composition.

Any foam agent may be employed, with the proviso that it does not deleteriously affect the antimicrobial efficacy of the composition. In one or more embodiments, the foam agent comprises a non-ionic foam agent such as decyl glucoside or an amphoteric foam agent such as cocamidopropylbetaine. In one or more embodiments, the amount of nonionic or amphoteric foam agent is from about 0.5 to about 3.5 wt. %, in other embodiments from about 1 to about 3 wt. %, based upon the total weight of the antimicrobial composition. In one or more embodiments, the amount of decyl glucoside or cocamidopropylbetaine is from about 0.5 to about 3.5 wt. %, in other embodiments from about 1 to about 3 wt. %, based upon the total weight of the antimicrobial composition.

Foam agents suitable for alcoholic compositions, i.e. where the amount of alcohol is greater than about 40 wt. %, include siloxane polymer surfactants, and are further described in co-pending U.S. Pat. App. Pub. No. 2007/0148101, which is hereby incorporated by reference in its entirety.

Examples of siloxane polymer surfactants include dimethicone PEG-7 undecylenate, PEG-10 dimethicone, PEG-8 dimethicone, PEG-12 dimethicone, perfluorononylethyl carboxydecal PEG 10, PEG-20/PPG-23 dimethicone, PEG-11 methyl ether dimethicone, bis-PEG/PPG-20/20 dimethicone, silicone quats, PEG-9 dimethicone, PPG-12 dimethicone, fluoro PEG-8 dimethicone, PEG 23/PPG 6 dimethicone, PEG 20/PPG 23 dimethicone, PEG 17 dimethicone, PEG5/PPG3 methicone, bis PEG20 dimethicone, PEG/PPG20/15 dimethicone copolyol and sulfosuccinate blends, PEG-8 dimethicone\dimmer acid blends, PEG-8 dimethicone\fatty acid blends, PEG-8 dimethicone\cold pressed vegetable oil \polyquaternium blends, random block polymers and mixtures thereof. In one or more embodiments, the antimicrobial composition comprises LAE, at least about 40 wt. % ethanol, a foam agent selected from PEG-10 dimethicone and PEG-12 dimethicone, and optionally 1,2-octanediol.

The amount of siloxane polymer foam agent is not particularly limited, so long as an effective amount to produce foaming is present. In certain embodiments, the effective amount to produce foaming may vary, depending upon the amount of alcohol and other ingredients that are present. In one or more embodiments, the antimicrobial composition includes at least about 0.002 wt. % of siloxane polymer foam agent, based upon the total weight of the antimicrobial composition. In another embodiment, the antimicrobial composition includes at least about 0.01 wt. % of siloxane polymer foam agent, based upon the total weight of the antimicrobial composition. In yet another embodiment, the antimicrobial composition includes at least about 0.05 wt. % of siloxane polymer foam agent, based upon the total weight of the antimicrobial composition.

In one embodiment, the foam agent is present in an amount of from about 0.002 to about 4 weight percent, based upon the total weight of the antimicrobial composition. In another embodiment, the foam agent is present in an amount of from about 0.01 to about 2 weight percent, based upon the total weight of the antimicrobial composition. It is envisioned that higher amounts may also be effective to produce foam. All such weights as they pertain to listed ingredients are based on the active level, and therefore, do not include carriers or by-products that may be included in commercially available materials, unless otherwise specified.

In other embodiments, it may be desirable to use higher amounts of foam agent. For example, in certain embodiments where the foaming antimicrobial composition of the present invention includes a cleansing or sanitizing product that is applied to a surface and then rinsed off, higher amounts of foam agent may be employed. In these embodiments, the amount of foam agent is present in amounts up to about 35 wt. %, based upon the total weight of the composition.

In one or more embodiments, the foam agent is added directly to the antimicrobial composition. In other embodiments, the foam agent is added to the antimicrobial composition as a solution or emulsion. In other words, the foam agent may be premixed with a carrier to form a foam agent solution or emulsion, with the proviso that the carrier does not deleteriously affect the foaming properties of the antimicrobial composition. Examples of carriers include any of the carriers described hereinabove for the cationic surfactant enhancers. It will be understood that, when the foam agent is premixed to form a foam agent solution or emulsion, the amount of solution or emulsion that is added to the antimicrobial composition may be selected so that the amount of foam agent falls within the ranges set forth hereinabove.

In certain embodiments, the antimicrobial composition of the present invention further includes at least one foam booster. In one embodiment, the foam booster comprises a cationic oligomer or polymer, a collagen amino acid, an amaranth protein, or a soluble elastin. Foam boosters are further described in co-pending U.S. patent application publication no. 2008/0207767, which is hereby incorporated by reference in its entirety.

The antimicrobial composition of the present invention may be formulated as an aerosol or non-aerosol foamable composition, and may be employed in any type of dispenser typically used for foam products. In one embodiment, the antimicrobial composition is used in dispensers that employ foaming pumps, which combine ambient air or an inert gas and the antimicrobial composition in a mixing chamber and pass the mixture through a mesh screen.

In one or more embodiments, the viscosity of the non-aerosol foamable composition is less than about 100 mPas, in one embodiment less than about 50 mPas, and in another embodiment less than about 25 mPas.

In one or more embodiments, the antimicrobial composition comprises a non-aerosol foamable alcoholic composition that includes LAE, greater than about 40 wt. % ethanol, based upon the total weight of the antimicrobial composition, and a siloxane polymer surfactant. In other embodiments, the antimicrobial composition comprises LAE, from about 2 to about 20 wt. % ethanol, and from about 0.5 to about 3.5 wt. % decyl glucoside, all based upon the total weight of the antimicrobial composition.

In one or more embodiments, the antimicrobial composition may be formulated as an antimicrobial gel. In these embodiments, the antimicrobial composition may comprise a thickener in addition to the LAE and select antimicrobial agent or preservative enhancer as described hereinabove.

In one embodiment, the antimicrobial composition includes one or more thickeners and optionally one or more stabilizers. Examples of thickeners and stabilizers include hydroxyethyl cellulose hydroxypropyl cellulose, methyl cellulose, carboxymethyl cellulose, and ammonium acryloyldimethyltaurate/VP copolymer. In one embodiment, where the thickener or stabilizer is starch-based, the thickener or stabilizer is present in an amount of up to about 10% by weight, in another embodiment in an amount of from about 0.1 to about 5% by weight, in yet another embodiment from about 0.2 to about 1% by weight, based upon the total weight of the antimicrobial composition. In other embodiments, where the thickener or stabilizer is a synthetic polymer, the thickener or stabilizer is present in an amount of up to about 15% by weight, in another embodiment in an amount of from about 0.1 to about 10% by weight, in yet another embodiment from about 1 to about 2% by weight, based upon the total weight of the antimicrobial composition.

In one or more embodiments, the antimicrobial composition may be thickened with polyacrylate thickeners such as those conventionally available and/or known in the art. Examples of polyacrylate thickeners include carbomers, acrylates/C 10-30 alkyl acrylate crosspolymers, copolymers of acrylic acid and alkyl (C5-C10) acrylate, copolymers of acrylic acid and maleic anhydride, and mixtures thereof.

In one or more embodiments, the polymeric thickener includes from about 0.5% to about 4% by weight of a cross-linking agent. Examples of cross-linking agents include the polyalkenyl polyethers.

Commercially available polymers of the polyacrylate type include those sold under the trade names Carbopol®, Acrysol® Polygel®, Sokalan®, Carbopol® 1623, Carbopol® 695, Ultrez 10, and Polygel® DB.

In one or more embodiments, the antimicrobial gel composition includes an effective amount of a polymeric thickener to adjust the viscosity of the antimicrobial gel to a viscosity range of from about 1000 to about 65,000 centipoise. In one embodiment, the viscosity of the antimicrobial gel is from about 5000 to about 35,000, and in another embodiment, the viscosity is from about 10,000 to about 25,000. The viscosity is measured by a Brookfield RV Viscometer using RV and/or LV Spindles at 22° C.+/−3° C.

As will be appreciated by one of skill in the art, the effective amount of thickener will vary depending upon a number of factors, including the amount of alcohol and other ingredients in the antimicrobial gel composition. In one or more embodiments, an effective amount of thickener is at least about 0.01 wt. %, based upon the total weight of the antimicrobial gel composition. In other embodiments, the effective amount is at least about 0.02 wt. %, in yet other embodiments at least about 0.05 wt. %, and it still other embodiments, at least about 0.1 wt. %. In one embodiment, the effective amount of thickener is at least about 0.5 wt. %, and in another embodiment, at least about 0.75 wt. %, based upon the total weight of the antimicrobial gel. In one or more embodiments, the compositions according to the present invention comprise up to about 10% by weight of the total composition of a polymeric thickener. In certain embodiments, the amount of thickener is from about 0.01 to about 1 wt. %, in another embodiment, from about 0.02 to about 0.4 wt. %, and in another embodiment, from about 0.05 to about 0.3 wt. %, based upon the total weight of the antimicrobial gel. In one embodiment, the amount of thickener is from about 0.1 to about 10 wt. %, in another embodiment from about 0.5% to about 5% by weight, in another embodiment from about 0.75% to about 2% wt. %, based upon the total weight of the antimicrobial gel.

In one or more embodiments, the antimicrobial gel may further comprise a neutralizer. Examples of neutralizing agents include amines, alkanolamines, alkanolamides, inorganic bases, amino acids, including salts, esters and acyl derivatives thereof. Examples of common neutralizers are further described in co-pending International Application Publication No. WO 2009/058802, which is hereby incorporated by reference.

The antimicrobial gel composition of the present invention may be employed in any type of dispenser typically used for gel products, for example pump dispensers. A wide variety of pump dispensers are suitable. Pump dispensers may be affixed to bottles or other free-standing containers. Pump dispensers may be incorporated into wall-mounted dispensers. Pump dispensers may be activated manually by hand or foot pump, or may be automatically activated. Useful dispensers include those available from GOJO Industries under the designations NXT® and TFX™ as well as traditional bag-in-box dispensers. Examples of dispensers are described in U.S. Pat. Nos. 5,265,772, 5,944,227, 6,877,642, 7,028,861, and U.S. Published Application Nos. 2006/0243740 A1 and 2006/0124662 A1, all of which are incorporated herein by reference. In one or more embodiments, the dispenser includes an outlet such as a nozzle, through which the antimicrobial gel composition is dispensed.

The antimicrobial composition may be prepared by simply mixing the components together. In one or more embodiments, the LAE is not added until after the other ingredients have been mixed, and the pH has been determined to be between about 3 to about 7. A pH adjuster may be employed if necessary. Buffers may also be employed.

In one embodiment, the antimicrobial gel composition is prepared by a method comprising dispersing the polymeric thickener in alcohol with slow to moderate agitation, adding water, and then adding any optional ingredients, determining the pH of the mixture and, if necessary, adjusting it to from about 3 to about 7, adding the LAE, and mixing until the mixture is homogeneous. As is known in the art, a neutralizer may be employed to neutralize the polymeric thickener and form the gel. A gel may be formed without a neutralizer if the thickener is one that swells when mixed with water or alcohol.

As described hereinabove, the antimicrobial composition of this invention includes a cationic surfactant such as LAE, and one or more of certain antimicrobial agents and preservatives. In one or more embodiments, the balance of the antimicrobial composition includes water or other suitable solvent. In one embodiment, one or more volatile silicone-based materials are included in the formulation to further aid the evaporation process. Exemplary volatile silicones have a lower heat of evaporation than alcohol. In certain embodiments, use of silicone-based materials can lower the surface tension of the fluid composition. This provides greater contact with the surface. In one embodiment, the silicone-based material, such as cyclomethicone, trimethylsiloxy silicate or a combination thereof, may be included in the formulation at a concentration of from about 4 wt. % to about 50 wt. % and in another embodiment from about 5 wt. % to about 35 wt. %, and in yet another embodiment from about 11 wt. % to about 25 wt. %, based upon the total weight of the antimicrobial gel composition.

The composition can further comprise a wide range of optional ingredients, with the proviso that they do not deleteriously affect the sanitizing efficacy of the composition. By deleterious is meant that the decrease in the log reduction according to the FDA TFM healthcare personnel hand wash test is not de minimus, or in other words, the log reduction does not decrease by more than about 0.5. The CTFA International Cosmetic Ingredient Dictionary and Handbook, Eleventh Edition 2005, and the 2004 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the present invention. Nonlimiting examples of functional classes of ingredients are described at page 537 of this reference. Examples of these functional classes include: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, humectants, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foam boosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein that are well known to one of ordinary skill in the art include solubilizing agents, sequestrants, keratolytics, topical active ingredients, and the like.

In certain embodiments, the antimicrobial composition comprises one or more humectants. Examples of humectants include propylene glycol, dipropyleneglycol, hexylene glycol, 1,4-dihydroxyhexane, 1,2,6-hexanetriol, sorbitol, butylene glycol, propanediols, such as methyl propane diol, dipropylene glycol, triethylene glycol, glycerin (glycerol), polyethylene glycols, ethoxydiglycol, polyethylene sorbitol, and combinations thereof. Other humectants include glycolic acid, glycolate salts, lactate salts, lactic acid, sodium pyrrolidone carboxylic acid, hyaluronic acid, chitin, and the like. In one embodiment, the humectant is present in an amount of from about 0.1 to about 20% by weight, based upon the total weight of the antimicrobial composition. In another embodiment the humectant is present in an amount of from about 1 to about 8% by weight, in another embodiment from about 2 to about 3% by weight, based upon the total weight of the antimicrobial composition.

In these or other embodiments, the antimicrobial composition comprises one or more conditioning or moisturizing esters. Examples of esters include cetyl myristate, cetyl myristoleate, and other cetyl esters, diisopropyl sebacate, and isopropyl myristate. In one embodiment, the ester is present in an amount of up to 10% by weight, based upon the total weight of the antimicrobial composition. In another embodiment the ester is present in an amount of from about 0.5 to about 5% by weight, in another embodiment from about 1 to about 2% by weight, based upon the total weight of the antimicrobial composition.

In one or more embodiments, the antimicrobial composition includes one or more emulsifying agents. Examples of emulsifying agents include stearyl alcohol, sorbitan oleate trideceth-2, poloxamers, and PEG/PPG-20/6 dimethicone. In one embodiment, the emulsifying agent is present in an amount of up to about 10% by weight, based upon the total weight of the antimicrobial composition. In another embodiment the emulsifying agent is present in an amount of from about 0.1 to about 5% by weight, in another embodiment from about 0.5 to about 2% by weight, based upon the total weight of the antimicrobial composition.

In one or more embodiments, the antimicrobial composition includes one or more solubilizers. Examples of solubilizers include PEG-40 hydrogenated castor oil, polysorbate-80, PEG-80 sorbitan laurate, ceteareth-20, oleth-20, PEG-4, and propylene glycol. The amount of solubilizer is not particularly limited, so long as it does not deleteriously affect the sanitizing efficacy of the composition.

In one or more embodiments, the antimicrobial composition includes one or more antiviral agents or antiviral enhancers. Examples of antiviral agents include botanicals such as rosmarinic acid, tetrahydrocurcuminoids, oleuropen, oleanolic acid, aspalathus linearis extract, white tea, red tea, green tea extract, neem oil limonoids, coleus oil, licorice extract, burnet, ginger & cinnamon extracts, alpha-glucan oligosaccharide, perilla ocymoides leaf powder, camphor, camellia oleifera leaf extract, ginger, menthol, eucalyptus, capillisil hc, hydroxyprolisilane cn, sandlewood oil/resin, calendula oil, rosemary oil, lime/orange oils, and hop acids. When used, the antiviral agents are present in amounts of from about 0.1 to about 1 percent by weight, based upon the total weight of the antimicrobial composition.

Examples of antiviral enhancers include proton donors, cationic oligomers and polymers, chaotropic agents, and copper and zinc compounds. Antiviral enhancers are further described in co-pending U.S. Patent Application Publications 2007/0184013, 2007/0185216, and 2009/0018213, all of which are hereby incorporated by reference.

Advantageously, certain ingredients that have been designated as critical to current antiseptic compositions can be limited in the antimicrobial composition of the present invention. For example, sulfones, antimicrobial metals, antibiotics, potassium sorbate, sodium sorbate, and sorbic acid can be limited, if desired, to less than about 0.5 wt. % each, or in another embodiment to less than about 0.1 wt. % each, based upon the total weight of the antimicrobial composition. In another embodiment, the antimicrobial composition is devoid of one or more of sulfones, antimicrobial metals, antibiotics, potassium sorbate, sodium sorbate, and sorbic acid.

As stated hereinabove, the antimicrobial composition of the present invention may be embodied in a variety of forms, including as a liquid, gel, or foam. In one embodiment, where the antimicrobial composition is in liquid form, the percent solids of the antimicrobial composition is less than about 6 percent, in another embodiment, less than about 5 percent, in yet another embodiment, less than about 4 percent, in still another embodiment, less than about 3 percent, in another embodiment, less than about 2 percent, in yet another embodiment, less than about 1 percent. The percent solids can be determined by various methods known in the art.

Unexpectedly, when LAE is combined with certain antimicrobial agents or preservatives according to the present invention, rapid antimicrobial activity is enhanced, i.e. potentiated. In one or more embodiments, the antimicrobial composition is effective in killing gram negative and gram positive bacteria, fungi, parasites, non-enveloped and enveloped viruses. In one or more embodiments, the antimicrobial composition has rapid antimicrobial efficacy against bacteria such as Staphylococcus aureus, methicillin-resistant S. aureus, Escherichia coli, Pseudomonas aeruginosa, Serratia marcescens, and fungi such as Candida albicans and Aspergillus niger. In one or more embodiments, the antimicrobial composition has rapid efficacy against skin microflora, including resident and transient skin microflora.

Thus, the present invention further provides a method for killing or inactivating microbes on a surface comprising applying, to the surface, an effective amount of an antimicrobial composition as described herein. Generally, an effective amount is an amount sufficient to contact the entire surface. The antimicrobial composition may be employed on a wide variety of surfaces or substrates, including skin, porous, and non-porous surfaces. The method provides a log reduction against a mixture of E. coli, S. aureus, Enterococcus faecium, and S. marcescens (Group 1) of at least 2, in some embodiments at least 3, in other embodiments at least 4, when tested at a contact time of about 15 seconds according to ASTM E 2783-10, Standard Test Method for Assessment of Antimicrobial Activity for Water Miscible Compounds Using a Time-Kill Procedure,” (ASTM International 2011) (formerly ASTM E 2315), said test method hereby incorporated by reference.

The method provides a log reduction against a mixture of S. aureus (MRSA), P. mirabilis, K. pneumoniae, and S. epidermidis (Group 2) of at least 2, in some embodiments at least 3, in other embodiments at least 4, when tested at a contact time of about 15 seconds according to ASTM E 2783-10.

The present invention further provides a method for inactivating viruses on a surface comprising applying, to the surface, an effective amount of an antimicrobial composition as described herein. The method provides a log reduction against Rotavirus and Influenza A of at least 2, in some embodiments at least 3, in other embodiments at least 4, when tested at a contact time of about 30 seconds according to ASTM 1052, “Standard Test Method for Efficacy of Antimicrobial Agents Against Viruses in Suspension” (ASTM International 2002), said test method hereby incorporated by reference.

It is envisioned that the antimicrobial composition of the present invention may be used as a healthcare personnel hand wash. It is expected that the present invention provides an antimicrobial composition that will meet the standards of the FDA Tentative Final Monograph for Healthcare Antiseptic Drug Products (TFM) (Federal Register 59 [116], Jun. 17, 1994: pp. 31402-31452) for healthcare personnel hand wash, said standard hereby incorporated by reference.

It is envisioned that the antimicrobial composition and method of the present invention will provide the sustained efficacy necessary to make them useful as surgical scrub compositions. Requirements for in vitro and in vivo testing of surgical hand scrubs are outlined in the FDA Tentative Final Monograph for Healthcare Antiseptic Drug Products (TFM) (Federal Register 59 [116], Jun. 17, 1994: pp. 31445-31448). The in vivo test procedure described beginning on page 31445 will hereinafter be referred to as the FDA TFM surgical hand scrub test. The antimicrobial efficacy of Surgical Scrubs can also be tested by any appropriate recognized test to demonstrate adequate disinfection of resident skin flora. Examples of such tests are ASTM E 1115-10, “Standard Test Method for Evaluation of Surgical Hand Scrub Formulations” (ASTM International 2010) and EN 12791:2005, “Chemical disinfectants and antiseptics, Surgical hand disinfection” (CEN-Comitée Européen de Normalisation, Brussels, Belgium), both of which test methods are incorporated by reference herein.

It is envisioned that the antimicrobial composition and method of the present invention will provide the rapid, broad-spectrum efficacy necessary to make the compositions useful as skin preparations as described and tested in ASTM E 1173-01 provides “Standard Test Method for Evaluation of Preoperative, Precatheterization, or Preinjection Skin Preparations” (ASTM International 2009) and FDA Tentative Final Monograph for Healthcare Antiseptic Drug Products (TFM) (Federal Register 59 [116], Jun. 17, 1994: pp. 31402-31452).

It is envisioned that the composition of the present invention will meet the standards of one or more of EN 1040:2005, entitled “Chemical disinfectants and antiseptics—Quantitative suspension test for the evaluation of basic bactericidal activity of chemical disinfectants and antiseptics” for basic bactericidal activity, EN 1275:2005, entitled “Chemical disinfectants and antiseptics—Quantitative suspension test for the evaluation of basic fungicidal or basic yeasticidal activity of chemical disinfectants and antiseptics” for basic fungicidal activity, EN 1500:1997, entitled “Chemical disinfectants and antiseptics—Hygienic handrub” for activity of products for use as a hygienic hand rub, EN 1499:1997, entitled “Chemical disinfectants and antiseptics—Hygienic handwash” for hygienic handwash, EN 14348:2005, entitled “Chemical disinfectants and antiseptics—Quantitative suspension test for the evaluation of mycobactericidal activity of chemical disinfectants in the medical area including instrument disinfectants” for tuberculoidal activity, EN 14476:2005+A1:2006, entitled “Chemical disinfectants and antiseptics—Virucidal quantitative suspension test for chemical disinfectants and antiseptics used in human medicine” for virucidal activity, and EN 12791:2005, entitled “Chemical disinfectants and antiseptics—Surgical hand disinfection” for surgical hand disinfection. All of the above standards are published by the European Committee for Standardization (CEN), and are incorporated by reference herein.

Advantageously, in one or more embodiments, the present invention further provides compositions and methods with rapid antimicrobial efficacy against gram positive and gram negative bacteria and fungi, as well as broad spectrum virucidal efficacy against one or more enveloped or one or more non-enveloped viruses. Examples of enveloped viruses include Herpes virus, Influenza virus; Paramyxovirus, Respiratory syncytial virus, Corona virus, HIV, Hepatitis B virus, Hepatitis C virus, SARS-CoV, and Toga virus. Non-enveloped viruses, sometimes referred to as “naked” viruses, include the families Picornaviridae, Reoviridae, Caliciviridae, Adenoviridae and Parvoviridae. Members of these families include Rhinovirus, Poliovirus, Adenovirus, Hepatitis A virus, Norovirus, Papillomavirus, and Rotavirus.

In order to demonstrate the practice of the present invention, the following examples have been prepared and tested. The examples should not, however, be viewed as limiting the scope of the invention. The claims will serve to define the invention.

### EXAMPLES

Examples 1-9 contained 10 wt. % ethanol in water. Examples 1-9 additionally contained 0.1 wt. % of a material known to have preservative efficacy, as summarized in the Table below. Example 9 (and all of the following examples containing LAE) was prepared by using Aminat-G, which is commercially available from Vedeqsa Inc. and which contains 20 wt. % LAE in glycerin. The pH of Examples 1-8 was within the recommended ranges for these preservatives. The pH of Example 9 was adjusted to between 7 and 9 by using sodium hydroxide.

In vitro efficacy of these compositions was measured against a mixture of E. coli, S. aureus, E. faecium, and S. marcescens (Group 1). The test was conducted according to the ASTM E 2315 method, “Standard Guide for Assessment of Antimicrobial Activity Using a Time-Kill Procedure,” which is also in accordance with ASTM E 2783-10, “Standard Test method for Assessment of Antimicrobial Activity for Water Miscible Compounds Using a Time-Kill Procedure.” Contact time was 15 seconds. Results are summarized in the table below. It can be seen that LAE exhibits surprising efficacy significantly greater than other preservatives.

TABLE 1

| | | | log₁₀ |
|---|---|---|---|
| | Ethanol | ENHANCER(s) | REDUCTION |
| EXAMPLE | (wt. %) | (0.1 wt. %) | GROUP 1 |
| 1 | 10 | quaternium-15 | 0.1 |
| 2 | 10 | phenoxyethanol | 0.0 |
| 3 | 10 | Potassium sorbate | 0.2 |
| 4 | 10 | Germaben II | 0.1 |
| 5 | 10 | Sodium benzoate | 0.0 |
| 6 | 10 | Merguard 1200 | 0.1 |
| 7 | 10 | Kathon CG | 0.2 |
| 8 | 10 | 3-iodoprop-2-ynyl N- | 0.1 |
| | | butylcarbamate (IPBC) | |
| 9 | 10 | LAE | >5.0 |

¹Germaben II - propylene glycol, propylparaben, methylparaben, and diazolidinyl urea from International Specialty Products
²Merguard 1200 - Methyldibromo Glutaronitrile (and) Phenoxyethanol from Nalco Company
³Kathon - 5-chloro-2-methyl-4-isothiazolin-3-one and 2-methyl-4-isothiazolin-3-one

Examples 10-13 contain 0.1 wt. % LAE. Examples 11 and 13 also contain 10 wt. % ethanol. Examples 12 and 13 were pH adjusted to about 5 using sodium hydroxide. In vitro efficacy of these compositions was measured against a mixture of S. aureus, and S. marcescens. The test was conducted as described above for Examples 1-9, with a contact time of 15 seconds. Results are summarized in the table below. For comparison purposes, log reduction for a 10 wt. % ethanol composition containing no LAE is less than 0.1 for both S. aureus and S. marcescens. It can be seen that ethanol enhances the efficacy of LAE. Surprisingly, the efficacy is greater at a higher pH of 5.

TABLE 2

| | | | | log₁₀ | log₁₀ |
|---|---|---|---|---|---|
| | | | | REDUCTION | REDUCTION |
| | LAE | Ethanol | | S. aureus | S. marcescens |
| EXAMPLE | (wt. %) | (wt. %) | PH | 33591 | 14756 |
| 10 | 0.1 | — | 3.5 | 0.5 | 2 |
| 11 | 0.1 | 10 | 3.5 | >5 | 4 |
| 12 | 0.1 | — | 5 | 2.5 | 3 |
| 13 | 0.1 | 10 | 5 | >5 | >5 |

Examples 14-28 contained 0.1 wt. % LAE and 0.4 wt. % glycerin (from Aminat-G). Examples 14-28 additionally contained one or more enhancers, as summarized in the Table below.

In vitro efficacy of these compositions was measured against a mixture of E. coli, S. aureus, E. faecium, and S. marcescens (Group 1). The test was conducted according to the procedures described above for Examples 1-9. Contact time was 15 seconds. Results are summarized in the table below. It can be seen that LAE exhibits surprising efficacy when certain enhancers are present.

Some data has shown that up to about 2-3 wt. % non-ionic and amphoteric surfactants can be used, and do not have a deleterious effect on antimicrobial efficacy. However, the data in Table 3 suggests that higher amounts of the non-ionic surfactant decyl glucoside and the amphoteric surfactant cocamidopropylbetaine have a negative effect on antimicrobial efficacy.

TABLE 3

| | | | log₁₀ |
|---|---|---|---|
| | LAE | ENHANCER(s) | REDUCTION |
| EXAMPLE | (wt. %) | (wt. %) | GROUP 1 |
| 14 | 0.1 | 10% ethanol | >5.0 |
| 15 | 0.1 | 5% ethanol | >5.0 |
| 16 | 0.1 | 0.3% Potassium sorbate | 1.8 |
| 17 | 0.1 | 0.5% Germaben II | 3.6 |
| 18 | 0.1 | 0.3% Sodium benzoate | 2.0 |
| 19 | 0.1 | 0.1% Merguard 1200 | 2.2 |
| 20 | 0.1 | 0.1% Kathon | 3.3 |
| 21 | 0.1 | 0.1% IPBC | 1.6 |
| 22 | 0.1 | 0.1% Quaternium-15 | >5.0 |
| 23 | 0.1 | 0.1% DMDM hydantoin | 2.3 |
| 24 | 0.1 | 0.6% Phenoxyethanol | >5.0 |
| 25 | 0.1 | 10% ethanol and | 0.9 |
| | | 4% cocamidopropylbetaine | |
| 26 | 0.1 | 10% ethanol and | 0.5 |
| | | 4% decyl glucoside | |
| 27 | 0.1 | 0.1% 3-iodoprop-2-ynyl N- | 1.6 |
| | | butylcarbamate (IPBC) | |

Efficacy was also measured against a mixture of S. aureus (MRSA), P. mirabilis, K. pneumoniae, and S. epidermidis (Group 2). The test was conducted according to the procedures described above for Examples 1-9. Contact time was 15 seconds. Examples 28-34 contained 0.1 wt. % LAE and 0.4 wt. % glycerin (prepared by using Aminat-G). Examples 29-34 additionally contained one or more enhancers, as summarized in the Table below. Examples 31-34 additionally contain 1 wt. % each of two natural foam extracts available from Active Organics under the tradenames Actiphyte® of Soap Wart and Actiphyte® of Soap Bark.

TABLE 4

| | | | log₁₀ |
|---|---|---|---|
| | LAE | ENHANCER(s) | REDUCTION |
| EXAMPLE | (wt. %) | (wt. %) | GROUP 2 |
| 28 | 0.1 | | 0.01 |
| 29 | 0.1 | 10% ethanol | >4.34 |
| 30 | 0.1 | 20% ethanol | >4.34 |
| 31 | 0.1 | | 1.24 |
| 32 | 0.1 | 10% ethanol | >4.38 |
| 33 | 0.1 | 20% ethanol | >4.38 |
| | | 3% decyl glucoside | |
| 34 | 0.1 | 0.5% caprylyl glycol | >4.38 |

Antiviral efficacy was tested according to ASTM 1052, “Standard Test Method for Efficacy of Antimicrobial Agents Against Viruses in Suspension” (ASTM International 2002). Compositions were tested at a 30 second exposure time, and 90% concentration. Example 35 was prepared from Aminat-G, SDA ethanol and water to contain 0.75 wt. % LAE, 3 wt. % glycerin, and 10 wt. % ethanol. Results are shown in the table below.

TABLE 5

| | Rotavirus | Influenza A |
|---|---|---|
| EXAMPLE | Log Reduction | Log Reduction |
| 35 | >5.13 | >4.50 |

Broad spectrum efficacy was tested against S. marcescens, Corynebacterium diptheriae, Enterococcus faecalis, Eschechia coli, Klebsiella pneumoniae, Acinetobacter baumannii, Pseudomonas aeruginosa, Salmonella cholerasius. Examples 36-38 contained 10 wt. % ethanol, 2 wt. % decyl glucoside, and varying amounts of LAE. Example 36 contained 0.1 wt. % LAE, Example 37 contained 0.25 wt. % LAE, and Example 38 contained 0.5 wt. % LAE. Examples 36-38 were adjusted to a pH of about 5.5. Example 39 was a commercially available handwash sold under the tradename Provon™ and containing about 3 wt. % triclosan.

Greater than 4 log reduction was achieved by Examples 36-38 against all organisms tested except Corynebacterium diptheriae, where greater than 3.5 log reduction was achieved. Example 39 achieved greater than 4 log reduction for all organisms except Corynebacterium diptheriae, greater than 3.5 log reduction for Corynebacterium diptheriae, but less than 2 log reduction for S. marcescens.

Pigskin samples were used in place of human hands to simulate the FDA Healthcare Personnel Handwash Test, Examples 39-43 contain 10 wt. % SDA ethanol, 3 wt. % glycerin, various amounts of LAE and shown in the table below, with the balance water.

Samples of fresh pig back fat with skin attached were obtained and cut into about 1.5 inch square. Fat was trimmed from the skin so that the samples were no more than 0.25 inches thick. The skin squares were washed with soap, rinsed with water, patted dry, and sanitized with 70% ethanol for about one minute. The skin squares were placed in a hydration chamber with glycerin and water overnight. The samples were inoculated with bacteria and allowed to dry for two minutes. Using a micropipette, 25 μL of the test product was applied to the center of the skin piece and rubbed in for about 30 seconds. The sample was allowed to dry for 2 minutes. Additional test product was added twice more, rubbed in, and dried. The samples were placed into 15 mL of Butterfield's phosphate buffered saline containing neutralizers (BPB+) and sonicated for 60 seconds to remove the bacteria from the skin sample. The solution was serially diluted and plated out according to standard procedures in the industry. The mean log reduction from baseline was determined, and is shown in Table 6 below. The “Control” sample is a commercially available product that has been shown to pass the FDA TFM test for healthcare personnel handwash, in which the active ingredient is 0.13 wt. % benzalkonium chloride.

TABLE 6

| | | Example 39 | Example 40 | Example 41 | Example 42 | Example 43 |
|---|---|---|---|---|---|---|
| | | 0.75 wt. % | 0.15 wt. % | 0.25 wt. % | 0.40 wt. % | 0.60 wt. % |
| Baseline | Control | LAE | LAE | LAE | LAE | LAE |
| 7.9 | 1.5 | 3.3 | 1.0 | 2.0 | 2.7 | 2.9 |
| 0.1 | 0.9 | 0.7 | 0.5 | 0.9 | 0.9 | 0.4 |

Compositions of the invention were tested according to the U.S. Environmental Protection Agenty (EPA) Sanitizer Test for Inanimate Surfaces, DIS/TSS-10 (1976). Examples 44-45 contained 10 wt. % SDA-3C ethanol, 1 wt. % glycerin, 0.25 wt. % LAE, 0.75 wt. % decyl glucoside, 0.015 wt. % lactic acid, and 0.50 wt. % sodium lactate. Example 44 was tested as a spray, while Example 45 was applied to an SMS wipe with about 500% loading. The samples were tested against Staphylococcus aureus ATCC 6538 and Klebsiella pneumoniae, aberrant, ATCC 4352, according to the EPA method. The contact time was 5 minutes. The results are shown in Table 7 below.

TABLE 7

| | % Bacterial Reduction | % Bacterial Reduction |
|---|---|---|
| EXAMPLE | Staphylococcus aureus | Klebsiella pneumoniae |
| 44 | >99.9998 | >99.9983 |
| 45 | >99.9998 | >99.9983 |

Various modifications and alterations that do not depart from the scope and spirit of this invention will become apparent to those skilled in the art. This invention is not to be duly limited to the illustrative embodiments set forth herein.

1. An antimicrobial composition comprising:
a cationic surfactant represented by the formula 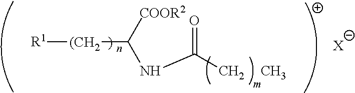
where R¹ is selected from 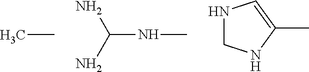
R² is an aromatic group or an alkyl group having from 1 to 18 carbon atoms, m is from about 8 to about 14, n is from 0 to about 4, X is chloride, bromide, or a counter ion derived from an organic or inorganic acid or a phenolic compound;
at least one of (i). from about 2 to about 90 wt. % of a C₁₋₆ alcohol, based upon the total weight of the antimicrobial composition, (ii). from about 0.02 to about 30 wt. % of a C₆₋₁₀ 1,2-alkanediol, based upon the total weight of the antimicrobial composition, and (iii). mixtures of (i) and (ii); wherein the pH of the antimicrobial composition is from about 3.5 to about 9.5.

2. The antimicrobial composition of claim 1, wherein X is a counter ion derived from acetic acid, citric acid, lactic acid, fumaric acid, maleic acid, gluconic acid, propionic acide, sorbic acid, benzoic acid, carbonic acid, glutamic acid, lauric acid, oleic acid, linoleic acid, phosphoric acid, nitric acid, sulfuric acid, or thiocyanic acid.

3. The antimicrobial composition of claim 1, wherein X is a counter ion derived from butylated hydroxyanisole (BHA), butylated hydroxytoluene, tertiary butylhydroquinone, methylparaben, ethylparaben, propylparaben, or butylparaben.

4. The antimicrobial composition of claim 1, wherein the pH of the antimicrobial composition is from about 5 to about 9.

5. The antimicrobial composition of claim 1, wherein the pH of the antimicrobial composition is from about 7 to about 8.

6. The antimicrobial composition of claim 1, wherein the antimicrobial composition comprises from about 0.02 to about 10 wt. % of a C₆₋₁₀ 1,2-alkanediol, based upon the total weight of the antimicrobial composition.

7. The antimicrobial composition of claim 6, wherein the C₆₋₁₀ 1,2-alkanediol is 1,2-hexanediol or 1,2-octanediol.

8. The antimicrobial composition of claim 7, wherein the C₆₋₁₀ 1,2-alkanediol is 1,2-octanediol.

9. The antimicrobial composition of claim 1, wherein the antimicrobial composition comprises from about 2 to about 20 wt. % of a C₁₋₆ alcohol, based upon the total weight of the antimicrobial composition.

10. The antimicrobial composition of claim 1, wherein the antimicrobial composition comprises from about 40 to about 90 wt. % of a C₁₋₆ alcohol, based upon the total weight of the antimicrobial composition.

11. The antimicrobial composition of claim 1, wherein the antimicrobial composition further comprises an antimicrobial agent or preservative selected from the group consisting of quaternary ammonium compounds, phenolic compounds, and 2-methyl-1,2-thiazol-3-one.

12. The antimicrobial composition of claim 11, wherein the antimicrobial agent or preservative comprises one or more of quaternium-15, benzalkonium chloride, 2-phenoxyethanol, methylparaben, ethylparaben, propylparaben, butyl paraben, sodium methyl paraben, sodium propyl paraben, butylparaben and isobutylparaben.

13. The antimicrobial composition of claim 1, wherein the antimicrobial composition further comprises a foam agent selected from siloxane polymer surfactants.

14. The antimicrobial composition of claim 1, wherein the antimicrobial composition further comprises a foam agent selected from non-ionic and amphoteric foam agents.

15. The antimicrobial composition of claim 14, wherein the non-ionic foam agent comprises decyl glucoside.

16. The antimicrobial composition of claim 1, wherein the antimicrobial composition further comprises a thickener.

17. The antimicrobial composition of claim 1, wherein the cationic surfactant is lauric arginate.

18. The antimicrobial composition of claim 1, wherein the antimicrobial composition comprises less than about 0.5 wt.

19. The antimicrobial composition of claim 1, wherein the antimicrobial composition comprises less than about 0.1 wt. %, based upon the total weight of the composition, of any of 2,4,4′-trichloro-2′-hydroxy-diphenylether (triclosan), 3,4,4-trichlorocarbanilid (triclocarban), 2-phenoxyethanol, chlorhexidine salts (CHG), parachlormetaxylenol (PCMX), hexetidine and cetylpyridinium salts.

20. The use of the composition of claim 1 as a surgical hand scrub when tested according to the FDA TFM surgical hand scrub test.

21. The use of the composition of claim 1 as a surgical hand scrub when tested according to standard test method EN 12791:2005.

22. The use of the composition of claim 1 as a surgical hand scrub when tested according to standard test method ASTM 1115-10.

23. The use of the composition of claim 1 as a preoperative, precatheterization, or preinjection skin preparation when tested according to standard test method ASTM 1173-01.

24. The use of the composition of claim 1 as a chemical disinfectant and antiseptic for basic bactericidal activity when tested according to standard test method EN 1040:2005

25. The use of the composition of claim 1 as a chemical disinfectant and antiseptic for basic fungicidal activity when tested according to standard test method EN 1275:2005.

26. The use of the composition of claim 1 as a hygienic hand rub when tested according to standard test method EN 1500:1997.

27. The use of the composition of claim 1 as a hygienic hand wash when tested according to standard test method EN 14348:2005.

28. The use of the composition of claim 1 as an instrument disinfectant when tested according to standard test method EN 14348:2005.

29. The use of the composition of claim 1 as a virucidal composition when tested according to standard test method EN 14476:2005.

30. A method for surface disinfection, the method comprising the steps of:
contacting a surface with an antimicrobial composition that includes LAE and an antimicrobial agent or preservative selected from C₁₋₆ alcohols, C₆₋₁₀ 1,2-alkanediols, and mixtures thereof, wherein the pH of the composition is from about 3.5 to about 9.5.

31. (canceled)

32. (canceled)

33. A method for disinfection of a porous or non-porous surface, the method comprising the steps of:
contacting a surface with an antimicrobial composition that includes LAE and an antimicrobial agent or preservative selected from C₁₋₆ alcohols, C₆₋₁₀ 1,2-alkanediols, and mixtures thereof, wherein the pH of the composition is from about 3.5 to about 9.5.
